(19) United States
(12) Patent Application Publication
(10) Pub. No.: US 2021/0217514 A1
(43) Pub. Date: Jul. 15, 2021

(54) SYSTEMS, METHODS, AND MEDIA FOR GENERATING PEER GROUP DRIVEN OPERATIONAL RECOMMENDATIONS

(71) Applicant: Skygen USA, LLC, Menomonee Falls, MI (US)

(72) Inventors: Russ Zimprich, Menomonee Falls, WI (US); Chakradhar Chekuri, Menomonee Falls, WI (US)

(21) Appl. No.: 17/148,002

(22) Filed: Jan. 13, 2021

Related U.S. Application Data
(60) Provisional application No. 62/960,394, filed on Jan. 13, 2020.

(51) Int. Cl. G16H 40/20; G06N 3/08

(52) U.S. Cl. CPC G16H 40/20; G06N 3/08

(57) ABSTRACT

In accordance with some embodiments of the disclosed subject matter, mechanisms (which can, for example, include systems, methods, and media) generating peer group driven operational recommendations for a dental practice is provided, the method comprising: receiving operations data associated with dental practices; training a model to identify causal relationships between outcomes and metrics derived from the operations data; associating the dental practice with a subset of dental practices exhibiting similar characteristics; generating, based on past outcomes, a suggested operational change for the dental practice likely to increase performance; presenting the suggested operational change; receiving updated operations data of the dental practice; determining that the dental practice is unlikely to sufficiently improve performance; in response, determining that the subset of dental practices is not the most appropriate group of dental practices; and associating the dental practice with a second subset that exhibits similar characteristics based on the updated operations data.

### CROSS-REFERENCE TO RELATED APPLICATIONS

This application claims the benefit of U.S. Provisional Patent Application No. 62/960,394, filed Jan. 13, 2020, which is hereby incorporated herein by reference in its entirety for all purposes.

### BACKGROUND

Many types of relatively small businesses, such as dental practices, physician practices, and the like, are run by proprietors that have little formal business training. Accordingly, such businesses can benefit from insights to improve the performance of their business from consultants that have more formal business training. However, the cost of such consultation can often be perceived as outweighing the benefits.

Accordingly, new systems, methods, and media for generating peer group driven operational recommendations are desirable.

### SUMMARY

In accordance with some embodiments of the disclosed subject matter, systems, methods, and media for generating peer group driven operational recommendations are provided.

In accordance with some embodiments of the disclosed subject matter, a method for generating peer group driven operational recommendations for a dental practice is provided, the method comprising: receiving operations data associated with a plurality of dental practices; training a computation model to identify causal relationships between outcomes and metrics derived from the operations data; associating the dental practice with a subset of dental practices of the plurality of dental practices that exhibit similar characteristics based on the operations data; generating, based on past outcomes associated with the subset of dental practices, a suggested operational change for the dental practice that is likely to increase performance of the dental practice; presenting the suggested operational change to a user associated with the dental practice; receiving updated operations data associated with the dental practice; determining, based on the updated operations data, that the dental practice is unlikely to sufficiently improve performance; in response to determining that the dental practice is unlikely to sufficiently improve performance, determining that the subset of dental practices is not the most appropriate group of dental practices; and associating the dental practice with a second subset of dental practices of the plurality of dental practices that exhibit similar characteristics based on the updated operations data.

In some embodiments, the operations data includes at least 10,000 values aggregated over.

In some embodiments, the computational model is a regression model with coefficients based on various performance metrics that are indicative of performance for one or more outcome categories.

In some embodiments, associating the dental practice with the subset of dental practices comprises: clustering the plurality of dental practices into a plurality of groups based on metrics derived from the operations data; and associating the dental practice with other dental practices in the same group of the plurality of groups as the dental practice.

In some embodiments, clustering the plurality of dental practices comprises utilizing a neural network to cluster the plurality of dental practices.

In some embodiments, each of the metrics derived from the operations data is associated with an outcome category of a plurality of outcome categories.

In some embodiments, the method further comprises: assigning each dental practice of the subset a scaled numerical score indicative of the respective dental practices performance in a first outcome category of the plurality of outcome categories based on a plurality of metrics associated with the first outcome category.

In some embodiments, the method further comprises presenting the scaled numerical score in a user interface element of a graphical user interface.

In some embodiments, the method further comprises: generating, based on past outcomes associated with the second subset of dental practices, a plurality of suggested operational changes for the dental practice that are likely to increase performance of the dental practice, each of the plurality of suggested operational changes associated with a particular increase in a scaled numerical score associated with a first outcome category of the plurality of outcome categories; and determining, for each suggested operational change of the plurality of suggested operational changes, a likelihood that the dental practice will achieve the particular increase in the scaled numerical score within a predetermined period of time; ranking the plurality of suggested operational changes based on the likelihood that the dental practice will achieve the particular increase in the scaled numerical score within the predetermined period of time; and presenting a subset of the ranked operational changes based on the ranking.

In some embodiments, the particular increase in the scaled numerical score is associated with a particular absolute improvement in one or more of the metrics associated with the first outcome category, and wherein determining the likelihood that the dental practice will achieve the particular increase in the scaled numerical score within the predetermined period of time comprises: identifying which of the subset of dental practices achieved the particular absolute improvement in the one or more metrics within a time period corresponding to the predetermined period based on the operations data associated with the subset of dental practices; and determining the likelihood based on the number of dental practices identified and the number of dental practices in the subset of dental practices.

In accordance with some embodiments of the disclosed subject matter, a system for generating peer group driven operational recommendations for a dental practice, the system comprising at least one processor that is configured to: receive operations data associated with a plurality of dental practices; train a computational model to identify causal relationships between outcomes and metrics derived from the operations data; associate the dental practice with a subset of dental practices of the plurality of dental practices that exhibit similar characteristics based on the operations data; generate, based on past outcomes associated with the subset of dental practices, a suggested operational change for the dental practice that is likely to increase performance of the dental practice; present the suggested operational change to a user associated with the dental practice; receive updated operations data associated with the dental practice; determine, based on the updated operations data, that the dental practice is unlikely to sufficiently improve performance; in response to determining that the dental practice is unlikely to sufficiently improve performance, determine that the subset of dental practices is not the most appropriate group of dental practices; and associate the dental practice with a second subset of dental practices of the plurality of dental practices that exhibit similar characteristics based on the updated operations data.

In accordance with some embodiments of the disclosed subject matter, a non-transitory computer readable medium containing computer executable instructions that, when executed by a processor, cause the processor to perform a method for generating peer group driven operational recommendations for a dental practice, the method comprising: receiving operations data associated with a plurality of dental practices; training a computational model to identify causal relationships between outcomes and metrics derived from the operations data; associating the dental practice with a subset of dental practices of the plurality of dental practices that exhibit similar characteristics based on the operations data; generating, based on past outcomes associated with the subset of dental practices, a suggested operational change for the dental practice that is likely to increase performance of the dental practice; presenting the suggested operational change to a user associated with the dental practice; receiving updated operations data associated with the dental practice; determining, based on the updated operations data, that the dental practice is unlikely to sufficiently improve performance; in response to determining that the dental practice is unlikely to sufficiently improve performance, determining that the subset of dental practices is not the most appropriate group of dental practices; and associating the dental practice with a second subset of dental practices of the plurality of dental practices that exhibit similar characteristics based on the updated operations data.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows an example of connections between various participants in a market for healthcare services.

FIG. 2 shows an example of a system for generating peer group driven operational recommendations in accordance with some embodiments of the disclosed subject matter.

FIG. 3 shows another example of a system for generating peer group driven operational recommendations in accordance with some embodiments of the disclosed subject matter.

FIG. 4 shows an example of hardware that can be used to implement a server and a computing device in accordance with some embodiments of the disclosed subject matter.

FIG. 5 shows an example of a process for generating and presenting peer group driven operational recommendations in accordance with some embodiments of the disclosed subject matter.

FIG. 6 shows an example of a process for providing feedback related to current performance in accordance with some embodiments of the disclosed subject matter.

FIG. 7 shows an example of a process for determining a likelihood of a provider achieving various outcomes based on previous actions of a peer group in accordance with some embodiments of the disclosed subject matter.

FIG. 8 shows an example of a process for generating operational recommendations for a particular provider in accordance with some embodiments of the disclosed subject matter.

FIG. 9 shows an example of a process for generating feedback to update a likelihood of a provider achieving various outcomes based on previous actions of a peer group and recent actions of the provider in accordance with some embodiments of the disclosed subject matter.

FIG. 10 shows an example of a user interface that can be used to present current scores indicative of the performance of a particular business in comparison to a group of peer businesses.

FIG. 11 shows an example of a user interface that can be used to receive new objectives, and present current objectives and associated probabilities of achieving the outcomes associated with the current objectives.

### DETAILED DESCRIPTION

In accordance with various embodiments, mechanisms (which can, for example, include systems, methods, and media) for generating peer group driven operational recommendations are provided.

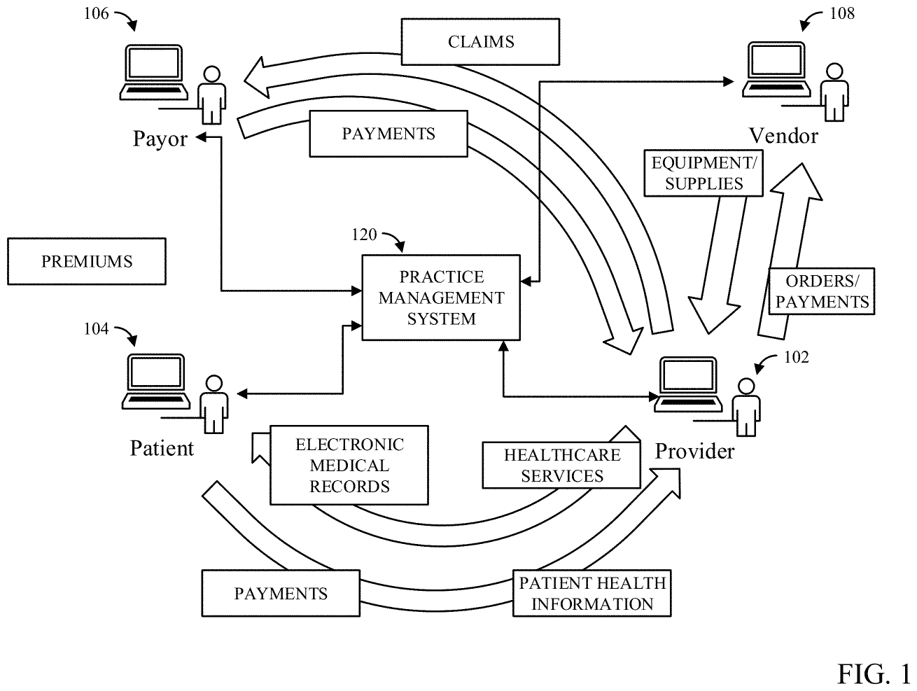

FIG. 1 shows an example of connections between various participants in a market for healthcare services and a system that can be implemented to manage one or more portions of a healthcare provider's practice in accordance with some embodiments of the disclosed subject matter. As shown in FIG. 1, a market for healthcare services can include various participants, such as a provider 102 of healthcare services, one or more patients 104, one or more payers 106, and one or more vendors 108. For example, provider 102 can be a private practice that includes a single healthcare provider, multiple healthcare providers that specialize in providing the same healthcare services, or multiple healthcare providers that have different specialties. Additionally or alternatively, provider 102 can be a clinic or hospital associated with a larger healthcare system. In a more particular example, provider 102 can be a practice that includes one or more dentists. As another more particular example, provider 102 can be a practice that includes one or more medical doctors. As yet another more particular example, provider 102 can be a practice that includes one or more optometrists and/or ophthalmologists. As still another example, provider 102 can be a practice that includes one or more psychiatrists, psychologists, and/or therapists.

As another example, patients 104 can be an existing patient of a particular provider 102, or a new patient of a particular provider 102. In some cases, a patient 104 may be associated with a payer to which premiums are paid by and/or on behalf of the patient.

As yet another example, payer 106 can be a private insurance company, a public insurance provider, and/or a government department and/or program. In some cases, payers 106 can negotiate reimbursement rates with individual providers. Additionally or alternatively, payers 106 can dictate reimbursement rates which individual providers can accept or reject. In some cases, payers 106 can include a publicly funded health plan (such as Medicare or Medicaid in the United States) which pay fixed fees for particular procedures to eligible providers on behalf of covered patients.

As still another example, vendor 108 can be a vendor of equipment and/or supplies. In a more particular example, vendor 108 can be a source of equipment that is used by dental practices, such as exam chairs, x-ray machines, sterilization equipment, hand pieces, etc. As another more particular example, vendor 108 can be a source of supplies used by dental practices, such as amalgam, anesthetic, bonding agents, gloves, etc.

In a market for healthcare services, participants can be linked by various transactions. For example, provider 102 can provide healthcare services to patient 104, and can receive payment and/or patient health information (e.g., protected health information as defined by the HIPAA Privacy Rule) from patient 102 in connection with the healthcare services. In some cases, provider 102 can provide patient 104 with access to the patient's electronic medical records. As another example, provider 102 can submit claims to payer 106 in connection with healthcare services provided to patient 104, and receive payment on the claims from payer 106. In such an example, payer 106 may or may not receive premiums from, and/or on behalf of, patient 104. As yet another example, provider 102 can submit orders for equipment and/or supplies to one or more of vendors 108, and can receive the ordered equipment and/or supplies from the corresponding vendor.

In some embodiments, a practice management system 120 can manage operations of one or more providers 102 and/or one or more of the interactions between participants in the market for healthcare services. For example, in some embodiments, practice management system 120 can provide a platform for scheduling patient visits, for managing employee work schedules, for maintaining electronic medical records, etc. As another example, practice management system 120 can be used to verify (e.g., with payer 106) a patients insurance status, submit claims to one or more payers 106, etc. As yet another example, practice management system 120 can be used to track equipment and/or supplies, facilitate discovery and/or ordering of equipment and/or supplies available from one or more vendors 108.

In some embodiments, practice management system 120 can use data about operations of a practice over time to generate recommendations of actions that a provider 102 can take to improve the performance of the provider's practice. Additionally or alternatively, in some embodiments, practice management system 120 can use data about operations of a practice over time to generate recommendations related to which equipment and/or supplies for the practice to order (e.g., based on implied or expressed preferences, prices, etc.).

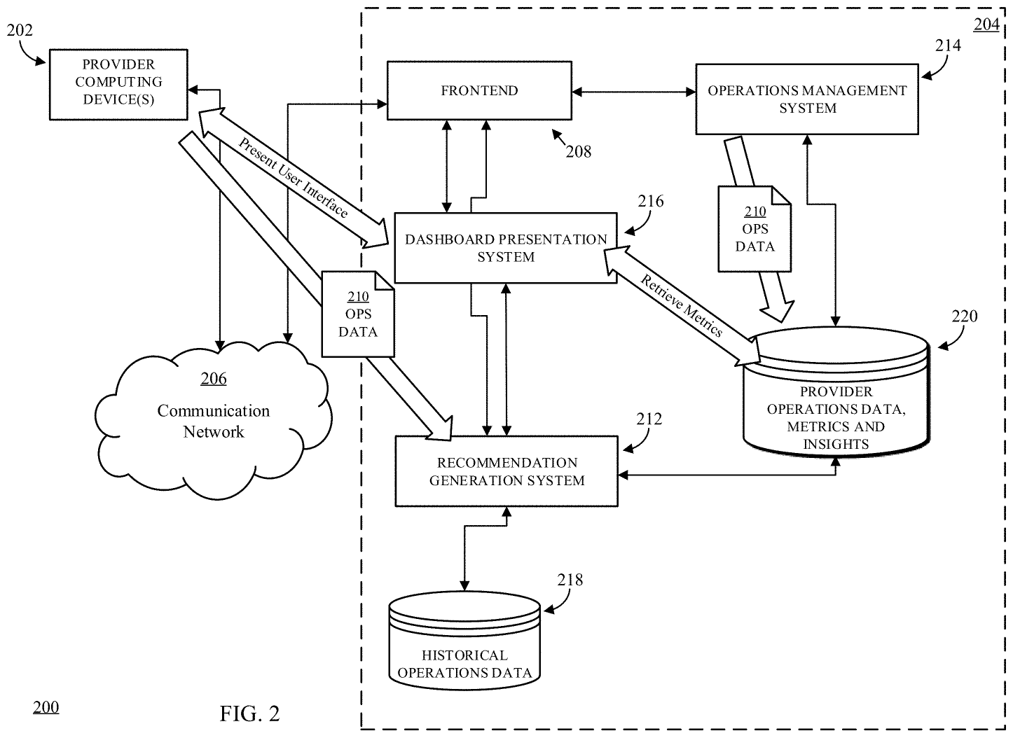

FIG. 2 shows an example 200 of a system for generating peer group driven operational recommendations in accordance with some embodiments of the disclosed subject matter. In some embodiments, system 200 can compute one or more metrics based on operations associated with a provider and can evaluate the metrics based on outcomes for other similar types of businesses (e.g., based on the size of the business and/or the type of business). For example, system 200 can generate one or more key performance indicators (KPI) that are metrics indicative of the performance of a particular business.

In some embodiments, system 200 can use a computational model to evaluate trends in the performance of a particular business to determine whether the current trend aligns with desired outcomes for that business. For example, if the trend(s) is in alignment with the desired outcomes, system 200 can update a probability that the desired outcome will be achieved within a certain period of time. As another example, if the trend(s) is not in alignment with the desired outcomes, system 200 can determine whether the particular business is associated with an appropriate group of peer businesses.

In some embodiments, if system 200 determines that the group of peer businesses is not appropriate, system 200 can determine if a more appropriate group of peer businesses exists and/or can be created. In some embodiments, system 200 can generate recommendations of actions to take for the particular to improve the performance of the business. In some embodiments, the recommendations can be based on a likelihood of the business performing to a particular outcome if the recommendation were implemented. The likelihood of each outcome can be based on the past performance of businesses in the group of peer businesses.

In some embodiments, system 200 can cause a user interface to be presented to a user associated with the particular business that includes recommendations and/or metrics related to the particular businesses performance in one or more areas and/or overall performance of the particular business. As shown in FIG. 2 in some embodiments, system 200 can include a computing device 202 associated with a particular user of a practice management service. In some such embodiments, the user can be a person (e.g., a medical practitioner, an office administrator, etc.) and/or an entity (e.g., a corporation, a non-profit organization, etc.). Additionally, in some embodiments, computing device 202 can act programmatically to perform one or more actions. In some embodiments, computing device(s) 202 can include one or more physical computing devices associated with a user (e.g., a personal computer, a laptop computer, a server, a smartphone, a tablet computer, a wearable computer, etc.), and virtual computing devices (e.g., provided via a compute service).

In some embodiments, computing device 202 can be part of a network of computing devices which can include one or more physical networks (e.g., which can be owned and/or operated by the user associated with computing device 202) and/or one or more virtual networks (e.g., which can be provided by physical computing devices made available by a service provider) including compute resources made available to the user through a compute service.

In some embodiments, computing device 202 can interact with a service for generating peer group driven operational recommendations that is provided, at least in part, by a computing environment 204 to transmit operations data to the service. For example, computing device 202 can submit a request to begin analyzing operations data associated with a particular provider. As another example, computing device 202 can submit a request for information indicative of performance of the provider. As yet another example, computing device 202 can submit instructions to the service indicating desired outcomes and/or to select recommended actions to pursue.

In some embodiments, communication network 206 can be any suitable wired network, wireless network, any other suitable network, or any suitable combination thereof. Additionally, communication network 206 can be any suitable personal area network, local area network, wide area network, over-the-air broadcast network (e.g., for radio or television), cable network, satellite network, cellular telephone network, any other suitable type of network, or any suitable combination thereof. For example, communication network 206 can include a publicly accessible network of linked networks, in some cases operated by various distinct parties, such as the Internet. In some embodiments, communication network 206 can include a private or semi-private network, such as a corporate or university intranet. Additionally, in some embodiments, communication network 206 can include one or more wireless networks, such as a Global System for Mobile Communications (“GSM”) network, a Code Division Multiple Access (“CDMA”) network, a Long Term Evolution (“LTE”) network, a 5G network, any other suitable wireless network, or any suitable combination of wireless networks. Communication network 206 can use any suitable protocols and/or components for communicating via the Internet and/or any of the other aforementioned types of networks. For example, communication network 206 can use one or more protocols or combinations or protocols, such as Hypertext Transfer Protocol (“HTTP”), HTTPS, Message Queue Telemetry Transport (“MQTT”), Constrained Application Protocol (“CoAP”), etc.

In some embodiments, among other things, frontend 208 can provide a user interface (e.g., a webpage, an application, etc.) that can be presented to a user of computing device 202, and the user can manually select and/or provide information that can be used to evaluate the performance of the provider associated with computing device 202. Additionally or alternatively, a user of computing device 202 can authorize the service to retrieve operations data generated by provider computing device 202, practice management system 120, and/or any other suitable computing device. In some embodiments, the service can receive operations data 210 (e.g., from computing device 202, practice management system 120, and/or any other suitable computing device), and can maintain one or more repositories that securely store operations data 210 and/or other operations data.

In some embodiments, system 200 can include a recommendation generation system 212 to receive, process, and/or analyze operations data 210 associated with one or more providers. In some embodiments, recommendation generation system 212 can use the operations data from the one or more providers to group providers into one or more peer groups, and generate recommendations for a particular provider based on the operations of other providers in the same peer group.

In some embodiments, frontend 208 can receive and process operations data from computing device 202 and/or any other suitable computing device. Frontend 208 can process messages received from computing device 202 and/or generated, for example, in response to events (e.g., when computing device 202 enters information into a user interface provided via frontend 208), and can determine whether the messages are properly authorized. For example, frontend 208 can determine whether a user and/or computing device associated with the message is authorized to request that changes be made to the service, and/or is authorized to grant permissions to others (e.g., recommendation generation system 212, an operations management system 214, etc.) to provide data related to operations of the provider associated with computing device 202 (e.g., operations data 210). In some embodiments, frontend 208 can include one or more APIs that can receive messages as API calls (e.g., from computing device 202 and/or any other suitable computing device). As such, in some embodiments, frontend 208 can effectuate one or more APIs for interacting with the service (and/or any portions thereof), such as one or more APIs for authorizing the service provider to retrieve and/or collect operations data (e.g., operations data 210) from any suitable computing device, providing operations data (e.g., operations data 210), etc.

In some embodiments, frontend 208 can be used to provide a user interface that is generated at least in part by a dashboard presentation system 216, which can use operations data associated with the provider to generate metrics that are indicative of the performance of the provider, generate user interface elements that are indicative of progress toward accomplishing a desired outcome, and/or any other suitable user interface elements.

In some embodiments, operations management system 214 can provide one or more operations management services to the provider, such services that allow the provider to schedule patients, schedule employees, compose invoices, track supplies and/or equipment, and/or any other tasks related to operations of the provider's practice. In some embodiments, operations management system 214 can be implemented as part of practice management system 120.

In some embodiments, system 200 can include a historical operations repository 218. In some embodiments, historical operations repository 218 can include operations data associated with one or more businesses that can be used to identify trends, associate trends with likely outcomes, determine actions that may be taken to achieve particular outcomes, and group businesses into peer groups. In some embodiments, operations data included in historical operations repository 218 can be de-identified such that identifying information of the business with which the data is associated cannot be easily ascertained from the data. For example, the size of the business can be generalized into a range, the location of the business can be generalized into a region, etc.

In some embodiments, system 200 can include a provider operations repository 220. In some embodiments, provider operations repository 220 can include operations data associated with a particular provider (e.g., the provider associated with computing device 202). Additionally, in some embodiments, provider operations repository 220 can be used to store metrics associated with the particular provider, desired outcomes for the provider, and/or insights/recommendations that have been generated for the particular provider. Alternatively, in some embodiments, such information can be stored in a separate repository (not shown).

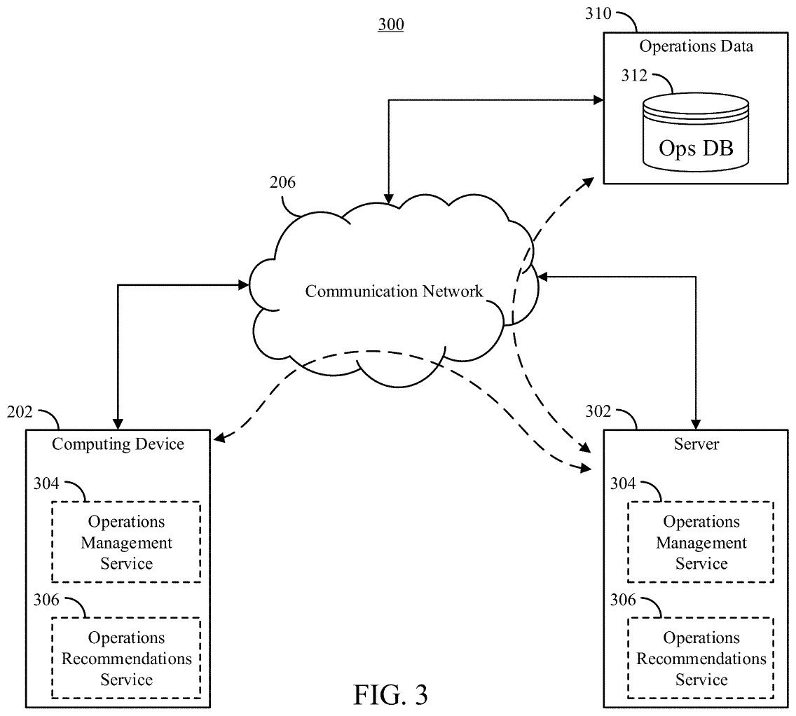

FIG. 3 shows another example 300 of a system for generating peer group driven operational recommendations in accordance with some embodiments of the disclosed subject matter. As shown in FIG. 3, a server (or other processing unit) 302 can execute one or more applications to provide access to an operations management service 304 (e.g., implemented by practice management system 120 and/or operations management system 214) and/or an operations recommendations service 306 (e.g., implemented by recommendation generation system 212). In some embodiments, operations management service 304 can facilitate day to day operations of a provider's practice, and operations recommendations service 306 can generate recommendations that can be acted upon to improve the performance (e.g., financial performance) of the provider's practice.

In some embodiments, server 302, operations management service 304, and/or operations recommendation service 306 can receive requests for information, queries, selections of items, user input, and/or any other suitable data, over communication network 206. In some embodiments, such information can be received from any suitable computing device, such as computing device 202. For example, computing device 202 can receive user input through an application being executed by computing device 202, such as through an input device (e.g., a keyboard, mouse, microphone, touchscreen, and the like). In such an example, computing device 202 can communicate information over communication network 206 to server 302 (or another server that can provide the information to server 302). As shown in FIG. 3, operations management service 304 and/or operations recommendations service 306 can be implemented using computing device 202 and/or server 302. For example, server 302 can be used to implement at least a portion of a back-end of operations management service 304 and/or operations recommendations service 306 and computing device 202 can be used to implement at least a portion of a front-end of operations management service 304 and/or operations recommendations service 306, such as a user interface.

In some embodiments, server 302 can communicate with one or more computing devices, such as an operations database server 310, to collect information regarding operations data associated with the provider (e.g., relating to operations of the provider's practice). In some embodiments, operations server 310 can be used (e.g., by the provider), to manage operations of a particular practice. For example, operations database server 310 can be used to manage a database 312 that includes information about a particular provider and/or practice. In some embodiments, server 302 can communicate with one or more operations database servers 310 to collect information about operations of a particular provider that is generated by an external practice management system/service (e.g., operations managed via a system/service other than operations management service 304).

In some embodiments, communications transmitted over communication network 206 and/or communication links shown in FIG. 3 can be secured using any suitable technique or combination of techniques. For example, in some embodiments, communications transmitted to and/or from server 302, computing device 202, and/or database server 310 can be encrypted using any suitable technique or combination of techniques. For example, communication between two or more computing devices associated with communication network 206 (e.g., server 302, computing device 202, database server 310, Domain Name System (DNS) servers, one or more intermediate nodes that serve as links between two or more other devices, such as switches, bridges, routers, modems, wireless access points, and the like) can be carried out based on Hypertext Transfer Protocol Secure (HTTPS). As another example, communications can be carried out based on Transport Layer Security (TLS) protocols and/or Secure Sockets Layer (SSL) protocols. As yet another example, communications can be carried out based on Internet Protocol Security (IPsec) protocols. As still another example, a virtual private network (VPN) connection can be established between one or more computing devices associated with communications network 206. In some embodiments, one or more techniques can be used to limit access to communication network 206 and/or a portion of communication network 206. For example, computing devices attempting to connect to the network and/or transmit communications using the network can be required to provide credentials (e.g., a username, a password, a hardware-based security token, a software-based security token, a one-time code, any other suitable credentials, or any suitable combination of credentials).

In some embodiments, one or more security techniques can be applied to any suitable portion of a communication network that interacts with computing devices. For example, security techniques can be used to implement a secure Wi-Fi network (which can include one or more wireless routers, one or more switches, and the like), a secure peer-to-peer network (e.g., a Bluetooth network), a secure cellular network (e.g., a 3G network, a 4G network, a 5G network, and the like, complying with any suitable standard(s), such as CDMA, GSM, LTE, LTE Advanced, WiMAX, 5G NR, and the like), and the like.

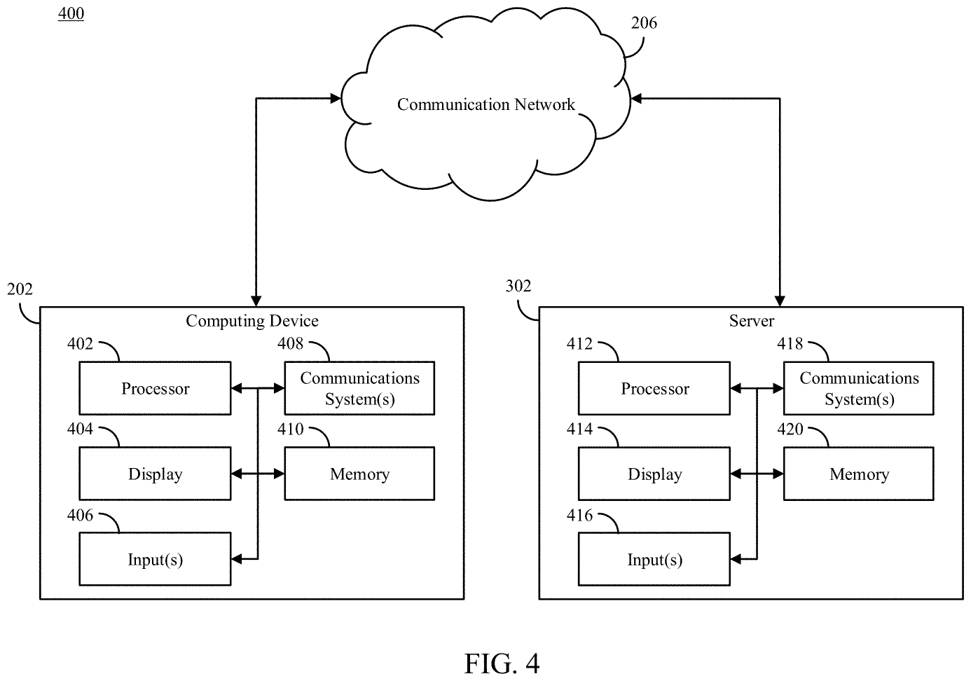

FIG. 4 shows an example 400 of hardware that can be used to implement server 302 and computing device 202 in accordance with some embodiments of the disclosed subject matter. As shown in FIG. 4, in some embodiments, computing device 202 can include a processor 402, a display 404, one or more inputs 406, one or more communication systems 408, and/or memory 410. In some embodiments, processor 402 can be any suitable hardware processor or combination of processors, such as a central processing unit (CPU), a graphics processing unit (GPU), and the like. In some embodiments, display 404 can include any suitable display devices, such as a computer monitor, a touchscreen, a television, and the like. In some embodiments, inputs 406 can include any suitable input devices and/or sensors that can be used to receive user input, such as a keyboard, a mouse, a touchscreen, a microphone, a camera, and the like.

In some embodiments, communications systems 408 can include any suitable hardware, firmware, and/or software for communicating information over communication network 206 and/or any other suitable communication networks. For example, communications systems 408 can include one or more transceivers, one or more communication chips and/or chip sets, and the like. In a more particular example, communications systems 408 can include hardware, firmware and/or software that can be used to establish a Wi-Fi connection, a Bluetooth connection, a cellular connection, an Ethernet connection, and the like.

In some embodiments, memory 410 can include any suitable storage device or devices that can be used to store instructions, values, and the like, that can be used, for example, by processor 402 to present content using display 404, to communicate with server 302 via communications system(s) 408, etc. Memory 410 can include any suitable volatile memory, non-volatile memory, storage, or any suitable combination thereof. For example, memory 410 can include RAM, ROM, EEPROM, one or more flash drives, one or more hard disks, one or more solid state drives, one or more optical drives, and the like. In some embodiments, memory 410 can have encoded thereon a computer program for controlling operation of computing device 202. In such embodiments, processor 402 can execute at least a portion of the computer program to present content (e.g., user interfaces, tables, graphics, and the like), receive content from server 302, transmit information to server 302, etc.

In some embodiments, server 302 can be implemented using one or more servers 302 (e.g., functions described as being performed by server 302 can be performed by multiple servers acting in concert) that can include a processor 412, a display 414, one or more inputs 416, one or more communications systems 418, and/or memory 420. In some embodiments, processor 412 can be any suitable hardware processor or combination of processors, such as a CPU, a GPU, etc. In some embodiments, display 414 can include any suitable display devices, such as a computer monitor, a touchscreen, a television, and the like. In some embodiments, inputs 416 can include any suitable input devices and/or sensors that can be used to receive user input, such as a keyboard, a mouse, a touchscreen, a microphone, and the like. In some embodiments, server 302 can be a mobile device.

In some embodiments, communications systems 418 can include any suitable hardware, firmware, and/or software for communicating information over communication network 206 and/or any other suitable communication networks. For example, communications systems 418 can include one or more transceivers, one or more communication chips and/or chip sets, etc. In a more particular example, communications systems 418 can include hardware, firmware and/or software that can be used to establish a Wi-Fi connection, a Bluetooth connection, a cellular connection, an Ethernet connection, and the like.

In some embodiments, memory 420 can include any suitable storage device or devices that can be used to store instructions, values, and the like, that can be used, for example, by processor 412 to present content using display 414, to communicate with one or more computing devices 202, etc. Memory 420 can include any suitable volatile memory, non-volatile memory, storage, or any suitable combination thereof. For example, memory 420 can include RAM, ROM, EEPROM, one or more flash drives, one or more hard disks, one or more solid state drives, one or more optical drives, etc. In some embodiments, memory 420 can have encoded thereon a server program for controlling operation of server 302. In such embodiments, processor 412 can execute at least a portion of the server program to transmit information and/or content (e.g., results of a database query, a portion of a user interface, textual information, graphics, etc.) to one or more computing devices 202, receive information and/or content from one or more computing devices 220, receive instructions from one or more devices (e.g., a personal computer, a laptop computer, a tablet computer, a smartphone, etc.), etc.

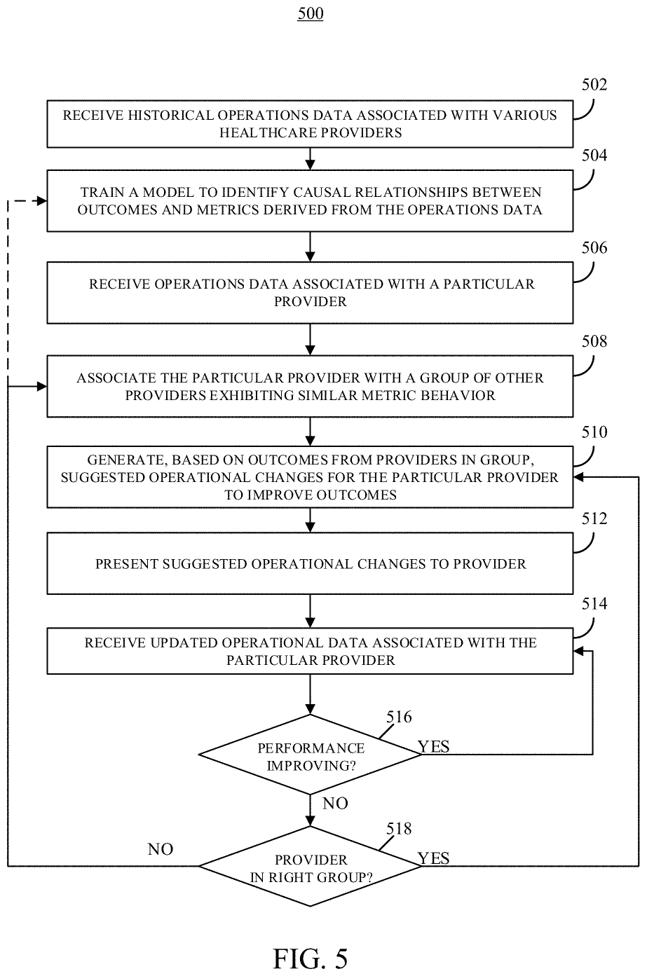

FIG. 5 shows an example 500 of a process for generating and presenting peer group driven operational recommendations in accordance with some embodiments of the disclosed subject matter. At 502, process 500 can receive historical operations data associated with various healthcare providers. In some embodiments, process 500 can receive the historical operations data from any suitable source, over any suitable period of time (e.g., weeks, months, years, etc.), and for any suitable number of healthcare providers (e.g., at least ten healthcare providers, dozens of health care providers, at least one hundred health care providers, hundreds of healthcare providers, etc.). For example, process 500 can receive operations data that has been collected in a data repository (e.g., historical operations repository 218). As another example, process 500 can receive operations data on an ongoing basis (e.g., from practice management system 120 and/or operations management system 214). As yet another example, process 500 can receive operations data from a computing device (e.g., computing device 202) associated with a particular provider. In such an example, a user of the computing device can export such operations data from an application used to manage operations of the provider. Even with relatively few healthcare providers, the volume of historical operations data quickly becomes unwieldy. For example, if process 500 receives historical operations data for twelve weeks from ten healthcare providers that see an average of 20 patients per day, such historical operations data associated with upwards of 2,500 patient visits (assuming 6 days of operation per week). As described below, each individual patient visit can be associated with multiple values, such as demographics associated with the patient, revenue, net revenue, billing information (e.g., how long it took to bill the patient/payer, how long it took to be paid by the patient/payer), etc., and information can be aggregated across patient visits. Accordingly, even for a small group of just ten relatively small practices over a relatively short period of time (e.g., about three months), the volume of data is relatively large (e.g., on the order of tens of thousands of values). As another example, process 500 can use at least 50,000 values from operational data to generate and present peer group driven operational recommendations. As described below, in such an example, the at least 50,000 values can be used to generate metrics, perform clustering, etc.

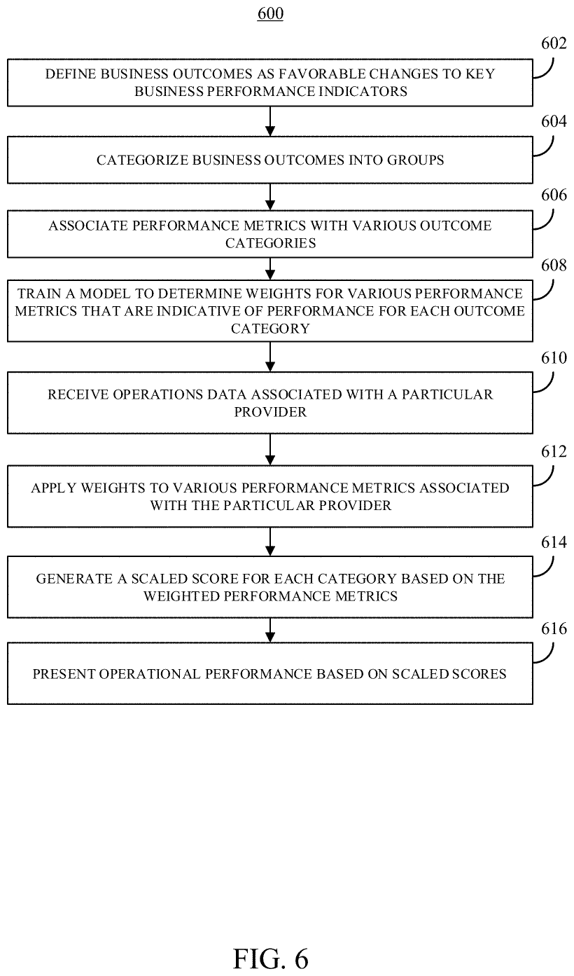

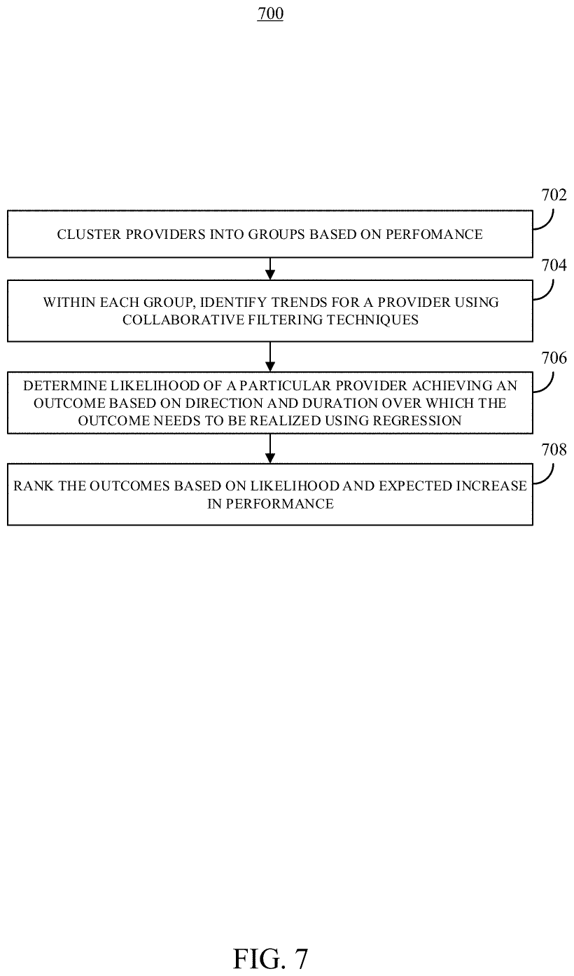

At 504, process 500 can train a model to identify causal relationship between outcomes and metrics derivable from operations data. In some embodiments, any suitable type of model can be used to identify causal relationships, such as models described below in connection with FIG. 6. In some embodiments, a model can be used to cluster businesses together, and for each cluster a model can be trained to identify causal relationships between outcomes and metrics for businesses in that cluster. In some embodiments, any suitable techniques or combination of techniques can be used to cluster businesses, such as techniques described below in connection with FIG. 7.

At 506, process 500 can receive operations data associated with a particular provider. In some embodiments, process 500 can receive the operations data from any suitable source and over any suitable period of time. For example, the operations data for the particular provider can be retrieved from a system and/or service used by the provider to manage operations of the provider's practice. As another example, the operations data can be provided by a user associated with the provider (e.g., via computing device 202). In a more particular example, the user can export operations data from an application used to manage operations of the particular provider.

At 508, process 500 can associate the particular provider with a group of other providers that exhibit similar metric behavior. In some embodiments, process 500 can use any suitable technique or combination of techniques to associate the particular provider with a peer group. For example, process 500 can use one or more filtering techniques to identify other providers that have similar characteristics (e.g., similar numbers of medical practitioners, similar numbers of employees, similarly sized markets, etc.). As another example, process 500 can use one or more clustering techniques to group the particular provider with a peer group. In a more particular example, one or more of k-means clustering, hierarchical clustering, mean-shift clustering, adaptive neural network techniques, auto encoders, and/or any other suitable clustering techniques can be used. Additionally, process 500 can use one or more clustering techniques described below in connection with FIG. 7.

In some embodiments, clustering at 508 can be performed by receiving initial clusters (e.g., based on an initial set of characteristics), and one or more clustering techniques can be used to modify the initial clusters.

In some embodiments, data can be received from a new provider (e.g., because a new provider has begun using a service associated with process 500). In such embodiments, the new provider can be associated with an existing cluster or clusters based on a determination of which cluster or clusters are most similar to the new provider. In such embodiments, the data from the new provider can be used when re-calibrating clusters (e.g., if the performance of providers that are following recommendations do not follow expectations as described below in connection with 516 and/or 518).

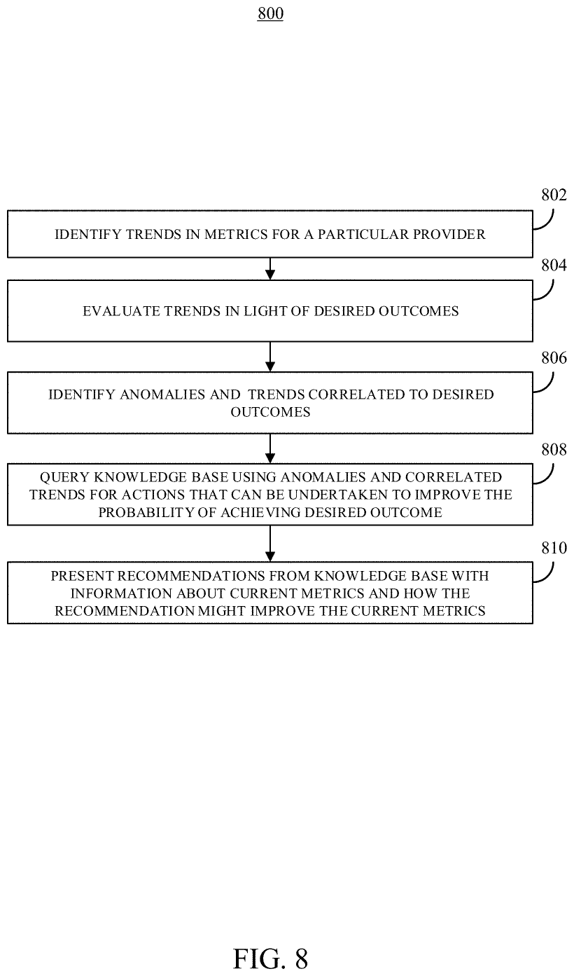

At 510, process 500 can generate suggested operational changes for the particular provider that are likely to improve outcomes. In some embodiments, the operational changes can be based on information derived from historical operations data of peer group providers. In some embodiments, process 500 can use any suitable technique or combination of techniques to generate suggested operational changes. For example, process 500 can compare metrics reflecting current and/or recent performance to historical values of the same metrics. In such an example, process 500 can determine how improvement in each of these metrics would impact the likelihood of the provider achieving one or more desired outcomes based on the trends and the performance of peer businesses. In some embodiments, process 500 can generate suggested operational changes using techniques described below in connection with FIGS. 7 and 8.

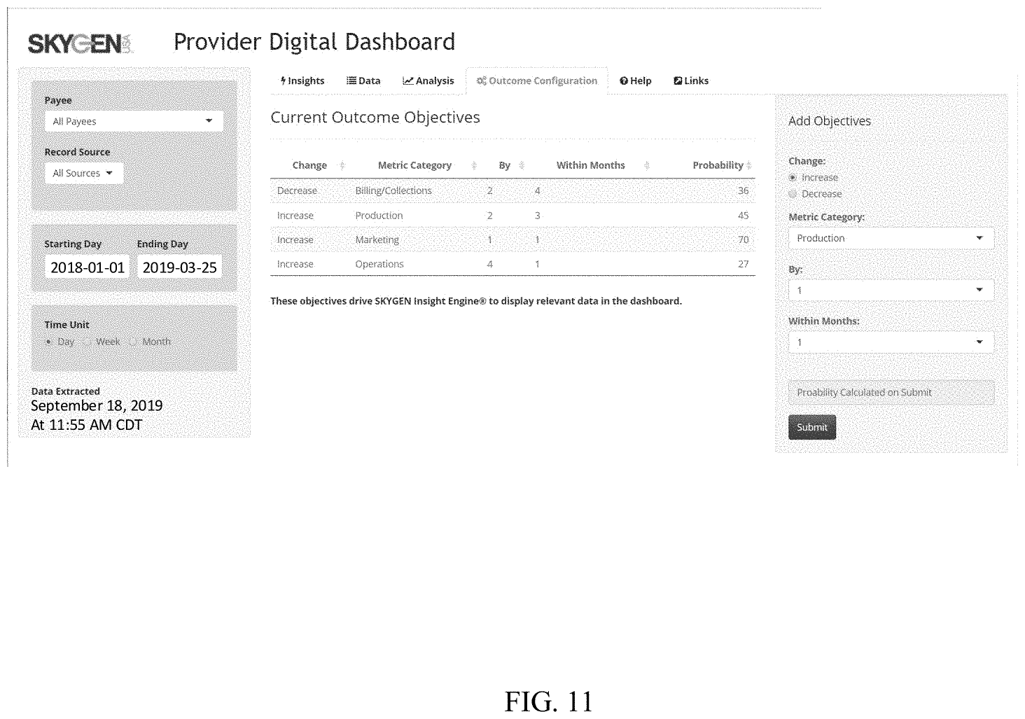

At 512, process 500 can present one or more suggested operational changes to the provider. In some embodiments, the suggested operational changes can be presented in connection with one or more desired outcomes that have been expressed by the provider (e.g., either implicitly or explicitly). For example, each suggested operational change can be associated with an area of performance which the provider has expressed an interest in improving. In a particular example, process 500 can use techniques described below in connection with FIGS. 8 and 11 to present suggested operational changes and/or information about desired outcomes that have been expressed by the provider.

At 514, process 500 can receive updated operational data associated with the particular provider. In some embodiments, process 500 can receive the updated operational data from any suitable source (e.g., as described above in connection with 506) and/or at any suitable time or times. For example, process 500 can receive updated operational data as it is generated and/or becomes available (e.g., from practice management system 120, from operations management system 214, etc.). As another example, process 500 can receive updated operational data at regular and/or irregular intervals.

At 516, process 500 can determine whether performance of the provider is improving and/or how much performance has improved within a particular period of time. If process 500 determines that performance has improved by an amount that is within expectations based on historical operations data and the updated operations data (“YES” at 516), process 500 can return to 514 and continue to receive updated operations data. Otherwise, if process 500 determines that performance has improved by an amount that is below expectations based on historical operations data and the updated operations data (“NO” at 516), process 500 can move to 518.

At 518, process 500 can determine whether the provider is still in the most appropriate group of peer businesses. In some embodiments, any suitable technique or combination of techniques can be used to determine whether the provider is in the most appropriate group. For example, process 500 can return to 508 after a predetermined period of time has passed (e.g., an interval of time since the business was last grouped with other businesses). As another example, if the performance of the provider is out of line with expectations based on the performance of peer group providers (e.g., performance may have exceeded expectations or underperformed expectations by a threshold amount), process 500 can determine that the provider is not in the most appropriate group of peer businesses. If process 500 determines that performance has improved by an amount that is within the most appropriate peer group (“YES” at 518), process 500 can return to 510 to generate new and/or updated suggested changes in light of the previously suggested changes not sufficiently improving the desired outcomes. Otherwise, if process 500 determines that the provider is no longer in the most appropriate group of peer businesses (“NO” at 518), process 500 can return to 508 and/or 504 and associate the provider with a new peer group and/or retrain the model to identify causal relationships between outcomes and metrics using additional operations data received subsequent to a previous training of the model.

FIG. 6 shows an example 600 of a process for providing feedback related to current performance in accordance with some embodiments of the disclosed subject matter. At 602, process 600 can define various business outcomes as corresponding to a change in a particular direction of a particular performance metric. Performance metrics can include any suitable objective matric that can be evaluated to determine a business's performance. For example, in a dental practice context, metrics can include one or more of monthly gross production, monthly net production, percentage of monthly gross production derived from fee for service (FFS) payments, percentage of monthly gross production based on payments made via a preferred provider organization (PPO) and/or a managed care organization, monthly overhead, and/or trends in one or more other metrics. As another example, in a dental practice context, metrics can include one or more of monthly net collections, monthly over-the counter collections, accounts receivable, and/or percentage of accounts receivable in various categories (e.g., 0-30 days, 31-60 days, etc.). As yet another example, in a dental practice context, metrics can include one or more of annual patient value, scheduled production for the current day (e.g., the day that the metric is being presented), scheduled production for one or more upcoming days (e.g., the next day, the next three days, etc.), mean production per visit, annual production per active patient, percent of total practice production (e.g., attributable to a particular provider), monthly treatment diagnosed (e.g., treatment recommended by a provider), and/or monthly treatment accepted (e.g., as a percentage of treatment diagnosed). As still another example, in a dental practice context, metrics can include one or more of new patients per month, number of visits in a particular time period, number of active patients (e.g., patients that have had a visit within a particular time period in the past), pre-appointment percentage, hygiene appointment percentage, and/or re-care. In some embodiments, business outcomes can be defined based on a positive change (e.g., a change expected to lead to increased profits) in one or more performance metrics. For example, process 600 can define a desired business outcome as a particular change in a particular metric. In a more particular example, process 600 can define a business outcome as an increase in the number of patients seen of 40 per month.

At 604, process 600 can categorize the business outcomes defined at 602 into various different groups that are generally uncorrelated to each other. For example, outcomes that are linked or correlated to each other can be placed into a group with each other, while outcomes that are not correlated can be placed into a different group (or excluded from all groups). For example, in the context of a dental practice, business outcomes can be categorized into categories such as production, billing and collections, operations and management, and marketing and messaging. In a particular example, business outcomes related to metrics such as monthly gross production, monthly net production, percentage of monthly gross production derived from FFS payments, percentage of monthly gross production based on payments made via a PPO and/or a managed care organization, monthly overhead, and/or trends in one or more other metrics can be categorized into a production category. As another more particular example, business outcomes related to metrics such as one or more of monthly net collections, monthly over-the counter collections, accounts receivable, and/or percentage of accounts receivable in various categories can be categorized into a billing and collections category. As yet another more particular example, business outcomes related to metrics such as one or more of annual patient value, scheduled production for the current day, scheduled production for one or more upcoming days, mean production per visit, annual production per active patient, percent of total practice production, monthly treatment diagnosed, and/or monthly treatment accepted can be categorized into an operations and management category. As still another more particular example, business outcomes related to metrics such as one or more of new patients per month, number of visits in a particular time period, number of active patients, pre-appointment percentage, hygiene appointment percentage, and/or re-care can be categorized into a marketing and messaging category. At 606, process 600 can group performance metrics into various outcome categories. In some embodiments, performance metrics can be grouped into categories using any suitable technique or combination of techniques. For example, in some embodiments, grouping can be based on input from one or more experts.

At 608, process 600 can train a model to determine weights for various performance metrics that are indicative of performance for the outcome categories defined at 604. In some embodiments, process 600 can determine the weights using any suitable technique or combination of techniques. For example, in some embodiments, weights for the various performance metrics can be generated by generating a regression model in which the weights are coefficients of the regression model. In some embodiments, weights can be based on performance data from members of a peer group. For example, past performance data from a peer group can be evaluated by process 600 to identify which performance metrics are most likely to have a causal impact on each outcome category. In some embodiments, as membership in a group changes and/or in response to receiving additional performance data, process 600 can update the weights (e.g., by running a new regression using updated data and/or data associated with the current membership of a group). In some embodiments, each group can be associated with a separate model, as different performance metrics may have variable effects for differently situated businesses. In some embodiments, the weights can be used to generate a metric associated with performance within a particular outcome category. For example, the weights can be applied to each performance metric in a particular outcome category to determine the overall performance in the outcome category with which the performance metrics are associated.

At 610, process 600 can receive operations data associated with a particular provider. In some embodiments, process 600 can receive the operations data from any suitable source (e.g., as described above in connection with 506 and/or 514 of FIG. 5) and/or at any suitable time or times.

At 612, process 600 can apply weights determined at 608 to various performance metrics associated with the particular provider. In some embodiments, the weights can be applied to a current value of each performance metric based on most recent operations data received at 610. In some embodiments, one or more of the performance metrics can be associated with a trend and/or a prediction. In such embodiments, a trend and/or prediction can be associated with a weight that can be used in calculating a score.

At 614, process 600 can generate a scaled score for each outcome category based on the weighted performance metrics. In some embodiments, the weighted performance metrics can be aggregated for each outcome category for each provider. In such embodiments, a resulting raw score (e.g., a non-scaled score) for the various providers in a group can be compared, and the providers can be ranked based on the raw scores, and associated with a scaled score based on the rank of the provider. For example, a lowest-scoring provider can be associated with a score of 0 on a scale of 0-10, while a highest-scoring provider can be associated with a score of 10. In such an example, providers with scores that fall between the highest scoring and lowest scoring can be given a scaled score using any suitable technique or combination of techniques. For example, a scaled score can be based on the rank of a particular business (e.g., one or more providers ranked behind the highest scoring provider can be associated with a scaled-score of 9, and one or more providers in a next highest ranked set of providers can be associated with a scaled-score of 8, and so on). As another example, a scaled score can be based on an interpolation of the raw score associated with a provider onto a scale based on the highest and lowest ranked providers. In a more particular example, a raw score can be mapped to a scaled score based on a linear interpolation between the lowest raw score form the group and the highest raw score from the group. In such examples, the scaled score can be provided as an integer (e.g., rounded to the nearest whole number). Alternatively, the scaled score can be provided to any suitable number of digits. In some embodiments, by scoring providers based on the standing of the provider with respect to members of the peer group, the scores can be relatively independent of positive and/or negative factors that affect many or all members of the peer group (e.g., a recession, an increase in reimbursement rates, a seasonal variation in the number of patients, etc.). In some embodiments, each of the scaled scores can be at the same scale to facilitate comparison between different categories.

At 616, process 600 can present operational performance characteristics to a user based on the scaled scores. In some embodiments, the operational performance characteristics can be presented using any suitable technique or combination of techniques. For example, in some embodiments, each category can be represented by one or more values (e.g., numbers and/or letters). As another example, in some embodiments, each category can be represented by a visualization (e.g., a dial, a gauge, stars or other symbols, a radar chart, etc.).

FIG. 7 shows an example 700 of a process for determining a likelihood of a provider achieving various outcomes based on previous actions of a peer group in accordance with some embodiments of the disclosed subject matter. At 702, process 700 can cluster providers into groups based on operations and performance characteristics. In some embodiments, process 700 can use any suitable characteristics to determine similarity between different providers and/or groups of providers. For example, process 700 can use the specialty, specialties, and/or lack of specialty (e.g., a general practice provider) of practitioners associated with a provider. As another example, process 700 can use location information, demographics of current patients, demographics of likely patients, demographics in an area around the practice, revenue, patient volumes, patient mix (e.g., a statistical distribution of one or more variables related to patients), etc. As yet another example, process 700 can use trends in performance metrics over time, how well the performance metrics of a particular business correlate with business outcomes, and/or current performance (e.g., based on standardized performance scores described above in connection with FIG. 6).

In some embodiments, process 700 can use any suitable technique or combination of techniques to cluster the providers. For example, process 700 can utilize one or more deep learning techniques to cluster providers of a certain business type. In a more particular example, process 700 can use an adaptive neural network and associated techniques to cluster providers of a certain business type. In another more particular example, process 700 can use an auto encoder and associated techniques to cluster providers of a certain business type. As another example, process 700 can use one or more statistical techniques to cluster providers of a certain business type. In a more particular example, statistical techniques can be include one or more of k-means clustering techniques, hierarchical clustering techniques, and/or mean-shift clustering techniques.

In some embodiments, process 700 can place each provider into a group that is determined to be most similar to the provider. For example, process 700 can use one or more of the clustering techniques described above to group providers into a number of groups such that providers in the same group are more alike with that group than with any other group. In a more particular example, a parameter that identifies an appropriate number (and/or maximum number) of clusters can be used to limit how many groups providers are divided into from a larger pool of providers. In such an example, the parameter can be used as a constraint to one or more of the clustering techniques described above.

In some embodiments, a single provider can be placed into multiple groups based on different characteristics. For example, a provider can be placed into different groups associated with different outcome categories. In a more particular example, a provider may be similar to one group for a first category of outcome (e.g., marketing), and a different group for another category of outcome (e.g., production).

At 704, process 700 can identify, within each group, trends correlated with improved outcomes. In some embodiments, process 700 can use any suitable technique or combination of techniques to identify trends correlated with improved outcomes. For example, process 700 can identify one or more providers in the same group that exhibit better performance in one or more outcome categories. In such an example, process 700 can compare the performance metrics of two or more providers to identify which metrics are correlated with the performance of the better performing provider.

At 706, process 700 can determine a likelihood of a particular provider achieving an outcome based on a direction and a duration over which the outcome needs to be realized based on a regression analysis of the length of time it took another business(es) to achieve the outcome in the past. In some embodiments, process 700 can determine a ratio of observed occurrences of a change of a particular magnitude in an outcome over a particular period of time compared to the number of possible occurrences. For example, process 700 can determine which of the providers in a group realized a particular change in a given time period, and calculate a probability based on a comparison of that number to the total number of providers in the group. In a more particular example, if there are 100 businesses in a group, and 10 of the businesses were observed to have realized a positive change of a predetermined magnitude (e.g., an amount that would cause a particular business to increase its scaled score by a particular amount) within a particular amount of time, process 700 can determine that there is a 10% likelihood that the particular business will be able to achieve that same change. In some embodiments, the period of time can be based on how quickly a metric can be expected to change, and can be on the order of weeks, months, or quarters. For example, a metric that historically changes relatively quickly for the group of providers can be associated with a shorter time period than a metric that changes more slowly.

At 708, process 700 can rank the outcomes based on the likelihoods determined at 706 and/or the expected increase in performance associated with the outcome. For example, if the likelihood of achieving an outcome is very high, but the increase in performance attributable to that outcome is low, the outcome may not be highly ranked regardless of the relatively high likelihood of achieving the outcome. In some embodiments, for each outcome category, outcomes can be presented to a user in a ranked order based on the likelihood that the outcome will increase performance.

FIG. 8 shows an example 800 of a process for generating operational recommendations for a particular provider in accordance with some embodiments of the disclosed subject matter. At 802, process 800 can identify trends in metrics for a particular provider. In some embodiments, process 800 can evaluate the metrics over any suitable period of time. For example, the period of time can be based on a historical rate of change for each metric across the group of providers, with metrics that historically change more rapidly being associated with shorter periods of time (e.g., days, or weeks), and metrics that change more slowly associated with longer periods of time (e.g., months, quarters, or years).

At 804, process 800 can evaluate trends in light of desired outcomes to determine whether the provider is likely to achieve desired outcomes. In some embodiments, process 800 can use any suitable to determine whether the provider is likely to achieve the desired outcomes. For example, process 800 can evaluate the rate of change of a particular metric, and can use past performance of other providers in the group to predict how the metric is likely to change in the future. In such an example, process 800 can identify other businesses in the same group that were otherwise in a similar position (e.g., as compared to others in the group) and that exhibited a similar rate of change in the past (e.g., the recent past), and use the performance of those businesses over a period of time to predict whether the current rate of change is sustainable.

At 806, process 800 can identify anomalies and trends that are correlated with the desired outcomes based on the evaluations at 804. In some embodiments, process 800 can use any suitable technique or combination of techniques to identify anomalies and/or trends correlated with desired outcomes. For example, in some embodiments, process 800 can determine an expected change and/or expected rate of change for one or more performance metrics based on operational data from providers in a particular group. In such an example, if a particular performance metric associated with a particular provider deviates by at least a threshold amount from the expected change and/or rate of change in the performance metric, that deviation can be identified as an anomaly. In some embodiments, anomalies can be positive or negative. For example, a particular provider may be improving in a particular metric more than expected (e.g., a positive anomaly), or less than expected (e.g., a negative anomaly). In some embodiments, in response to identifying a negative anomaly, process 800 can prompt the provider associated with the negative anomaly to review whether the provider is following recommendations that have previously been selected (e.g., by a user associated with the provider) in connection with the performance metric that is associated with a negative anomaly and/or prompt the user to review recommendations that may help the provider improve performance.

At 808, process 800 can query a knowledge base using the anomalous and/or correlated trends to retrieve actions that if undertaken are predicted to increase the probability of achieving desired outcomes. In some embodiments, the knowledge base can include information about any suitable entity, such as models, clusters, providers, variables used in the models, business outcomes, metrics (e.g., performance metrics), and/or any other suitable entities. In some embodiments, the knowledge base can include any suitable information about entities. For example, the knowledge base can include information about entities (e.g., providers), relationships between entities, changes observed over time, statistical inferences related to changes to entities and relationships between entities. In some embodiments, the knowledge base can be a relational data store that can be configured to be queried using any suitable query language(s).

At 810, process 800 can present recommendations for actions to undertake based on results received from the knowledge base. In some embodiments, process 800 can present information about current metrics and/or scores (e.g., normalized scores), and how implementing one or more of the recommendations can be expected to improve the current metrics and/or scores (e.g., normalized scores).

In some embodiments, one or more portions of process 800 can be repeated for each peer group in which the provider is a member. For example, as describe above in connection with FIG. 7, a provider can be associated with a peer group for each category of outcome

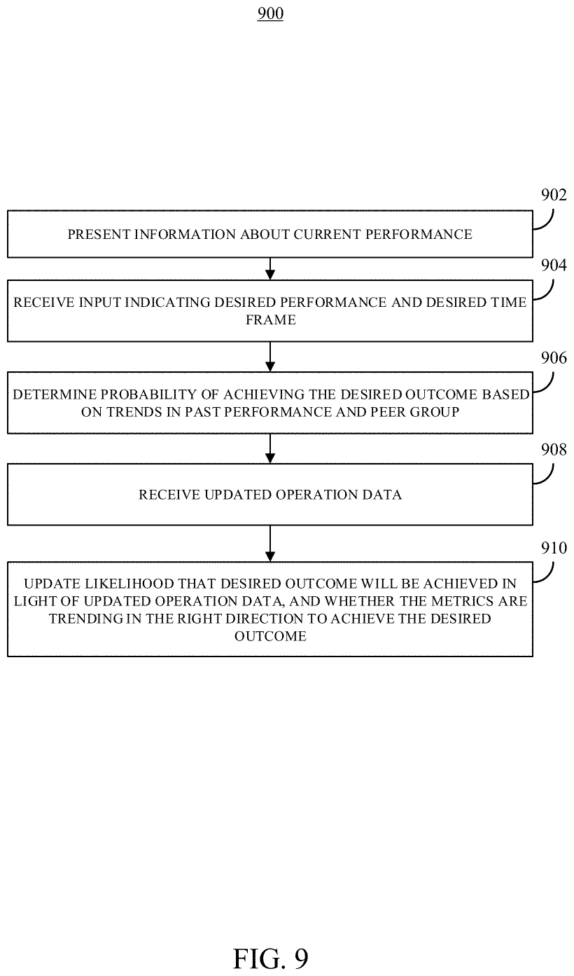

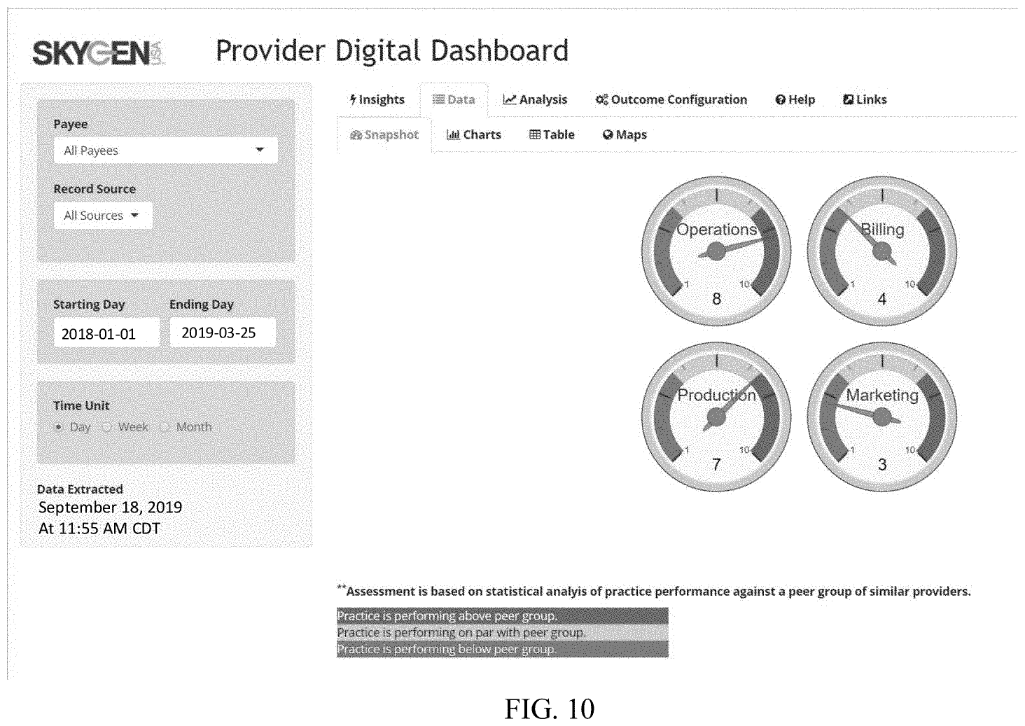

FIG. 9 shows an example 900 of a process for generating feedback to update a likelihood of a provider achieving various outcomes based on previous actions of a peer group and recent actions of the provider in accordance with some embodiments of the disclosed subject matter. At 902, process 900 can present information about a provider's current performance in one or more outcome categories and/or one or more metrics. In some embodiments, process 900 can present the information using any suitable technique or combination of techniques. For example, in some embodiments, process 900 can present the information as a dashboard interface. As described above, in some embodiments, process 900 can present information about current performance using standardized scores (e.g., from 1-10). For example, process 900 can present information about current performance using a user interface shown in FIG. 10. In some embodiments, a user can request presentation of at least a portion of the underlying data that was used to generate the score(s), such as performance metrics associated with various outcomes.

At 904, process 900 can receive input indicating one or more desired performance improvements and/or one or more desired time frames in which to improve performance. In some embodiments, process 900 can receive input indicating that a user has a preference for improving performance in a particular outcome category and/or for an amount of improvement (e.g., improvement to a scaled-score that is 1 point higher than the current scaled score). Additionally, in some embodiments, process 900 can receive input indicating a time period over which the improvement is to be realized. In some embodiments, any suitable user interface can be used to receive input, such as the user interface shown in FIG. 11.

At 906, process 900 can determine a probability of the provider achieving the desired outcome based on trends in past performance and/or peer group performance. For example, as described above in connection with 706 of FIG. 7, process 900 can determine, based on trends in the provider's performance and/or in the performance of the peer group, a probability of the provider successfully achieving the desired outcome within the specification time period. Additionally or alternatively, process 900 can determine a probability of the provider successfully achieving the desired outcome within various specific time periods in connection with presenting a user interface for receiving input about a desired time over which the desired outcome is to be achieved. For example, the probability can be calculated for various time periods (e.g., one month, 3 months, 6 months, etc.), and can be presented in a user interface for receiving the desired time frame in connection with selectable user interface elements for receiving input to select a desired time frame. In some embodiments, the probability that a particular outcome can be achieved can be determined using any suitable technique or combination of techniques, such as techniques described above in connection with 706 of FIG. 7.

At 908, process 900 can receive updated operation data. In some embodiments, updated operations data can be received from any suitable source (e.g., as described above in connection with 506 and/or 514 of FIG. 5) and/or at any suitable time or times. For example, process 900 can receive updated operational data as it is generated and/or becomes available (e.g., from practice management system 120, from operations management system 214, etc.). As another example, process 900 can receive updated operational data at regular and/or irregular intervals.

At 910, process 900 can update a likelihood that the desired outcome will be achieved in light of the updated operations data, and whether the metrics are trending in the right direction to achieve the desired outcome. In some embodiments, the likelihood that the desired outcome will be achieved can be determined using any suitable technique or combination of techniques, such as techniques described above in connection with 706 of FIG. 7. For example, the probability can change from 906 to 910 because the value of the performance metrics and the period of time over which the outcome is to be achieved can change, thus changing the magnitude of the change required to achieve the desired outcome.

FIG. 10 shows an example of a user interface that can be used to present current scores indicative of the performance of a particular business in comparison to a group of peer businesses. As shown in FIG. 10, various outcome categories (e.g., operations, billing, production, and marketing) can each be associated with a graphical user interface element that represents a scaled-score associated with the outcome category. In some embodiments, additional graphical elements (e.g., color coding in FIG. 10) can be used to demonstrate context for the score (e.g., whether the provider being evaluated is above, below, or on par with members of the peer group of providers being used to generate the scores). As described above, the scaled-scores can change over time as the performance of the provider changes.

FIG. 11 shows an example of a user interface that can be used to receive new objectives, and present current objectives and associated probabilities of achieving the outcomes associated with the current objectives. As shown in FIG. 11, a first portion of a user interface can present objectives that have previously been submitted, and a probability of achieving the objectives. Another portion of the user interface can be used to receive user input to add a new objective, which can include user interface elements for specifying an outcome category to change, a direction of change, a magnitude of change in scaled score, and/or a period of time over which the change is to be accomplished. In some embodiments, a provider can choose to try to reduce performance in one or more outcome categories. For example, a provider can choose to reduce performance in one or more categories in which reducing performance would have a large impact on cost while having a relatively low impact on profitability. In general, mechanisms described herein can attempt to predict/recommend actions/efforts to increase overall profitability based on measured changes to various business metrics. It should be noted that, as used herein, the term mechanism can encompass hardware, software, firmware, or any suitable combination thereof.

It should be understood that the above-described steps of the processes of FIGS. 5-9 can be executed or performed in any order or sequence not limited to the order and sequence shown and described in the figures. Also, some of the above steps of the processes of FIGS. 5-9 can be executed or performed substantially simultaneously where appropriate or in parallel to reduce latency and processing times.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways.

What is claimed is:

1. A method for generating peer group driven operational recommendations for a dental practice, the method comprising:
receiving operations data associated with a plurality of dental practices;
training a computational model to identify causal relationships between outcomes and metrics derived from the operations data;
associating the dental practice with a subset of dental practices of the plurality of dental practices that exhibit similar characteristics based on the operations data;
generating, based on past outcomes associated with the subset of dental practices, a suggested operational change for the dental practice that is likely to increase performance of the dental practice;
presenting the suggested operational change to a user associated with the dental practice;
receiving updated operations data associated with the dental practice;
determining, based on the updated operations data, that the dental practice is unlikely to sufficiently improve performance;
in response to determining that the dental practice is unlikely to sufficiently improve performance, determining that the subset of dental practices is not the most appropriate group of dental practices; and
associating the dental practice with a second subset of dental practices of the plurality of dental practices that exhibit similar characteristics based on the updated operations data.

2. The method of claim 1, wherein the operations data includes at least 10,000 values aggregated over.

3. The method of claim 1, wherein the computational model is a regression model with coefficients based on various performance metrics that are indicative of performance for one or more outcome categories.

4. The method of claim 1, wherein associating the dental practice with the subset of dental practices comprises:
clustering the plurality of dental practices into a plurality of groups based on metrics derived from the operations data; and
associating the dental practice with other dental practices in the same group of the plurality of groups as the dental practice.

5. The method of claim 4, wherein clustering the plurality of dental practices comprises utilizing a neural network to cluster the plurality of dental practices.

6. The method of claim 1, wherein each of the metrics derived from the operations data is associated with an outcome category of a plurality of outcome categories.

7. The method of claim 6, further comprising:
assigning each dental practice of the subset a scaled numerical score indicative of the respective dental practices performance in a first outcome category of the plurality of outcome categories based on a plurality of metrics associated with the first outcome category.

8. The method of claim 7, further comprising presenting the scaled numerical score in a user interface element of a graphical user interface.

9. The method of claim 6, further comprising:
generating, based on past outcomes associated with the second subset of dental practices, a plurality of suggested operational changes for the dental practice that are likely to increase performance of the dental practice, each of the plurality of suggested operational changes associated with a particular increase in a scaled numerical score associated with a first outcome category of the plurality of outcome categories; and
determining, for each suggested operational change of the plurality of suggested operational changes, a likelihood that the dental practice will achieve the particular increase in the scaled numerical score within a predetermined period of time;
ranking the plurality of suggested operational changes based on the likelihood that the dental practice will achieve the particular increase in the scaled numerical score within the predetermined period of time; and
presenting a subset of the ranked operational changes based on the ranking.

10. The method of claim 9, wherein the particular increase in the scaled numerical score is associated with a particular absolute improvement in one or more of the metrics associated with the first outcome category, and
wherein determining the likelihood that the dental practice will achieve the particular increase in the scaled numerical score within the predetermined period of time comprises:
identifying which of the subset of dental practices achieved the particular absolute improvement in the one or more metrics within a time period corresponding to the predetermined period based on the operations data associated with the subset of dental practices; and
determining the likelihood based on the number of dental practices identified and the number of dental practices in the subset of dental practices.

11. A system for generating peer group driven operational recommendations for a dental practice, the system comprising at least one processor that is configured to:
receive operations data associated with a plurality of dental practices;
train a computational model to identify causal relationships between outcomes and metrics derived from the operations data;
associate the dental practice with a subset of dental practices of the plurality of dental practices that exhibit similar characteristics based on the operations data;
generate, based on past outcomes associated with the subset of dental practices, a suggested operational change for the dental practice that is likely to increase performance of the dental practice;
present the suggested operational change to a user associated with the dental practice;
receive updated operations data associated with the dental practice;
determine, based on the updated operations data, that the dental practice is unlikely to sufficiently improve performance;
in response to determining that the dental practice is unlikely to sufficiently improve performance, determine that the subset of dental practices is not the most appropriate group of dental practices; and
associate the dental practice with a second subset of dental practices of the plurality of dental practices that exhibit similar characteristics based on the updated operations data.

12. The system of claim 11, wherein the operations data includes at least 10,000 values aggregated over.

13. The system of claim 11, wherein the computational model is a regression model with coefficients based on various performance metrics that are indicative of performance for one or more outcome categories.

14. The system of claim 11, wherein the at least one processor is further configured to:
cluster the plurality of dental practices into a plurality of groups based on metrics derived from the operations data; and
associate the dental practice with other dental practices in the same group of the plurality of groups as the dental practice.

15. The system of claim 14, wherein the at least one processor is further configured to:
cluster the plurality of dental practices utilizing a neural network to cluster the plurality of dental practices.

16. The system of claim 11, wherein each of the metrics derived from the operations data is associated with an outcome category of a plurality of outcome categories.

17. The system of claim 16, wherein the at least one processor is further configured to:
assign each dental practice of the subset a scaled numerical score indicative of the respective dental practices performance in a first outcome category of the plurality of outcome categories based on a plurality of metrics associated with the first outcome category.

18. The system of claim 17, wherein the at least one processor is further configured to:
present the scaled numerical score in a user interface element of a graphical user interface.

19. The system of claim 16, wherein the at least one processor is further configured to:
generate, based on past outcomes associated with the second subset of dental practices, a plurality of suggested operational changes for the dental practice that are likely to increase performance of the dental practice, each of the plurality of suggested operational changes associated with a particular increase in a scaled numerical score associated with a first outcome category of the plurality of outcome categories; and
determine, for each suggested operational change of the plurality of suggested operational changes, a likelihood that the dental practice will achieve the particular increase in the scaled numerical score within a predetermined period of time;
rank the plurality of suggested operational changes based on the likelihood that the dental practice will achieve the particular increase in the scaled numerical score within the predetermined period of time; and
present a subset of the ranked operational changes based on the ranking.

20. The system of claim 19, wherein the particular increase in the scaled numerical score is associated with a particular absolute improvement in one or more of the metrics associated with the first outcome category, and
wherein the at least one processor is further configured to:
identify which of the subset of dental practices achieved the particular absolute improvement in the one or more metrics within a time period corresponding to the predetermined period based on the operations data associated with the subset of dental practices; and
determine the likelihood based on the number of dental practices identified and the number of dental practices in the subset of dental practices.

21. A non-transitory computer readable medium containing computer executable instructions that, when executed by a processor, cause the processor to perform a method for generating peer group driven operational recommendations for a dental practice, the method comprising:
receiving operations data associated with a plurality of dental practices;
training a computational model to identify causal relationships between outcomes and metrics derived from the operations data;
associating the dental practice with a subset of dental practices of the plurality of dental practices that exhibit similar characteristics based on the operations data;
generating, based on past outcomes associated with the subset of dental practices, a suggested operational change for the dental practice that is likely to increase performance of the dental practice;
presenting the suggested operational change to a user associated with the dental practice;
receiving updated operations data associated with the dental practice;
determining, based on the updated operations data, that the dental practice is unlikely to sufficiently improve performance;
in response to determining that the dental practice is unlikely to sufficiently improve performance, determining that the subset of dental practices is not the most appropriate group of dental practices; and
associating the dental practice with a second subset of dental practices of the plurality of dental practices that exhibit similar characteristics based on the updated operations data.
